(19) United States
(12) Patent Application Publication
(10) Pub. No.: US 2022/0163546 A1
(43) Pub. Date: May 26, 2022

(54) Methods and Compositions for Self-Administration of a Drug

(71) Applicant: Frederic J. HUSER, Greenwich, CT (US)

(72) Inventor: Frederic J. HUSER, Greenwich, CT (US)

(21) Appl. No.: 17/529,787

(22) Filed: Nov. 18, 2021

Related U.S. Application Data
(63) Continuation-in-part of application No. 16/773,309, filed on Jan. 27, 2020.
Application No. 16/773,309 is a continuation of application No. 14/860,248, filed on Sep. 21, 2015, now abandoned.
Application No. 14/860,248 is a continuation of application No. 13/771,704, filed on Feb. 20, 2013, now abandoned.
(60) Provisional application No. 61/601,308, filed on Feb. 21, 2012.

(51) Int. Cl. G01N 33/92

(52) U.S. Cl. CPC G01N 33/92

(57) ABSTRACT

A method of administering an over the counter drug for treatment of a chronic medical condition to a patient is provided wherein a drug label for over-the-counter treatment of a chronic medical condition is created which requires a new patient to take an initial point-of-service diagnostic test to determine whether the patient suffers from the condition treated by the drug and requires patients already receiving the drug on an over-the-counter basis to take one or more follow up point-of-service diagnostic tests to determine whether the patient is benefiting from the drug and are not experiencing undue side effects. Patients determined eligible to take the drug may then be provided with a random access code for use in association with a lockable, electromechanical cabinet by means of which the patient can receive an appropriate dose of the drug to treat the patient's chronic medical condition. In certain embodiments, a follow up point-of-service diagnostic test determines whether the patient is experiencing any undue side effects from the drug. In further embodiments, a national patient registry for containing the point of service diagnostic test results is created for access by appropriate health care professionals.

### FIELD OF THE INVENTION

The present invention relates to the regulatory change in status of certain prescription (“Rx”) chronic care drugs dispensed only on a doctor's order to over-the-counter (“OTC”) sale only with the assistance of retail pharmacy personnel based on point-of-sale diagnostic data to both ensure for the individual patient that the drug is effective for the labeled use and also to prevent/block the sale of the drug for both off-guideline labeled use as well as for off-label use. It is directed to the following combination of elements: 1) a condition-of-use drug label requiring as a condition of sale a pharmacy-assisted CLIA-waived diagnostic test with real-time results in less than 30 minutes (i.e., the FDA approved label on the drug's package is a combination product label [term of regulatory art] which designates one and only one companion diagnostic test to be used and which requires healthcare personnel (e.g., pharmacy personnel) to take certain diagnostic actions with the companion diagnostic generating certain minimum/maximum biomarker results as specified in the drug label in order for the pharmacy personnel to be able to determine the patient's suitability and to be legally able to offer the drug for sale to an individual patient. It should be noted that the companion diagnostic test is cross-labeled with the same information that is on the drug label indicating that its use is a mandatory condition for the pharmacy to be able to make an initial and second sale or no sale decision on the drug); 2) the diagnostic actions will be administered only at the point of sale (“POS”) in Food, Drug, or Mass Merchandiser stores which have Clinical Laboratories Improvement Act (“CLIA”) certificates of waiver from the appropriate state and/or federal regulatory body to conduct CLIA-waived diagnostic tests with trained healthcare personnel present (such as those in a pharmacy) and will be conducted by retail store healthcare personnel to determine before drug use the presence of the chronic condition and to determine after drug use drug effectiveness and safety in the individual patient; and 3) a patient registry accessible by participating pharmacies, which enables pharmacy personnel, rather than the patient, to record the data from each patient who took the diagnostic test as specified in the drug's label so that as a matter of convenience the patient, if eligible, can purchase refills of the drug in question from any other participating retailer. It should be noted that the data in the patient registry is only referenced as proof that the patient has taken all the mandatory tests and that the individual patient is, therefore, eligible or not to repurchase the drug.

### BACKGROUND OF THE INVENTION

Drug labeling refers to all of the printed information that accompanies a drug, including the label, the wrapping and the package insert. In the United States, drug labeling is regulated by the U.S. Food and Drug Administration (“FDA”). These regulations apply to prescription drugs, over-the-counter (nonprescription) drugs, and dietary supplements. The drug label must include instructions for correctly using the medicine, including the dosage and what to do if the patient misses a dose. This description should cover any special instructions such as taking the medicine with food or water. In addition, the label should spell out storage instructions and general instructions, such as discussing questions with a health professional, because the label is not intended to be all-inclusive.

As a subset of drug labels, there is a type called a combination product drug label. In general this type of label currently refers only to an Rx drug, which as a condition of use must be used in combination with either a particular device or diagnostic test. The device or diagnostic test is cross-labeled so that it is clear to healthcare providers that the device or diagnostic test is tied to the use of the drug and that the drug is tied to the use of the device or diagnostic test. As a result, neither the drug nor the device/diagnostic test can be used independently until the conditions of use have been met. (Herceptin for breast cancer carries such a combination drug label with a specified diagnostic test). All such combination labels heretofore approved by the FDA are for Rx drugs only and have never included a CLIA-waived diagnostic test as the companion diagnostic test. Only non-CLIA-waived diagnostic tests have been included in such Rx drug labels, which is indicative of how sophisticated these types of Rx diagnostic tests are. In the invention described herein the methods describe for the first time an OTC combination drug label, which requires the use of a CLIA-waived POS diagnostic test, which is easily readable by moderately trained retail healthcare personnel. Therefore, the pertinent industry to consider is not the Rx drug industry but rather the OTC drug industry.

While it is considered desirable to allow for OTC access to certain medications while ensuring proper usage thereof, safety remains a serious concern. In particular, Rx-to-OTC switches of chronic care drugs have often been tricky to accomplish because of potential safety issues no matter how carefully the label is constructed. Nevertheless, such switches may help patients keep certain chronic disease states or conditions under better or more convenient control while also cutting healthcare costs with respect to physician visits and prescription filling, and may also present an opportunity for increased revenue for retail establishments (such as pharmacies). These types of switches so far have only related to diseases with signs and symptoms that can be self-diagnosed.

However, FDA has had serious concerns about safety and whether patients can be trusted to self-diagnose, self-purchase, and use chronic care drugs, e.g., for asymptomatic conditions on an unsupervised OTC basis. One example of a long-standing need for an Rx-to-OTC switch involves cholesterol drugs such as lovastatin and pravastatin, which are prescription only (“Rx”) “statins” used to reduce cholesterol in patients with elevated cholesterol levels. For approximately two decades, pharmaceutical companies such as Merck and Bristol Myers Squibb have been attempting to switch Mevacor® (lovastatin) and Pravachol® (pravastatin), respectively, from Rx status to OTC status. Their efforts have included attempts to design special drug labels as part of the packaging which would, in theory, enable patients with elevated cholesterol to understand how to correctly self-select, self-purchase and self-treat with these drugs with minimal healthcare provider involvement. The companies have asserted that drug labels can be developed which are suitable for the OTC purchase environment that would enable consumers to determine whether the drug was appropriate for them to take on an OTC basis or whether the patient's condition was unsuitable for self-diagnosis and self-treatment. Critical to this self-diagnosis and self-treatment paradigm has been the ability of the drug's label to communicate to some patients in a retail setting without the aid of a healthcare professional that they should not purchase and use the drug and therefore should de-select themselves from the relevant patient population. The desire is that the consumer would know in advance of the purchase decision their correct cholesterol levels and to evaluate that information in the context of the drug's label without the aid of a healthcare professional informing them they should or should not purchase and use the drug.

Despite pharmaceutical manufacturers' best efforts to conduct so-called Labeling Comprehension and Actual Use Studies to convince regulators that a drug's label can be used to communicate all relevant information for an asymptomatic condition in an OTC setting to both appropriate and inappropriate patients, the FDA has denied the approval of all chronic care “Rx-to-OTC” New Drug Application (“NDA”) submissions for such disease conditions on multiple occasions because it has been determined that patients are simply not compliant, meaning that they either don't know their correct biometric data or do not interact with a healthcare practitioner to obtain the needed biometric data before making the purchase/repurchase decision and may be exposing themselves to serious side effects. Reportedly, Merck alone has spent over $100,000,000 during a fifteen-year period attempting to switch Mevacor to OTC status three times hoping labeling alone would communicate the necessary information and instructions and that patients would be compliant.

The FDA's general concern with lack of sufficient patient compliance with the instructions in the label is related to “off-guideline labeled use.” In the case of statins, as an example, which can cause serious adverse events such as liver damage and muscle damage (rhabdomyolysis), the FDA does not want patients who do not have sufficiently elevated levels of cholesterol using them. This can lead to a situation where the patient is receiving very little or no benefit from using the statin but is exposed to all the risks in the form of side effects. Obviously, if patients are misremembering what their cholesterol levels are, or are not making any attempt to see their physician for a diagnostic test and are estimating their biometric data, this patient behavior increases the odds of creating a scenario where the risk of over-using the drug outweighs the benefit.

As an example of real-world compliance, the following quote appears in an article on a recent failed Pfizer clinical study to be used to switch Lipitor to OTC status: “A 1,200-subject actual use trial to simulate the OTC use of Lipitor 10 mg did not meet its primary objectives of demonstrating patient compliance with the direction to check low-density lipoprotein cholesterol (“LDL-C”) level and after checking their LDL-C level take appropriate action based on test results.” (The Tan Sheet: By Malcolm Spicer/Email the Author/View Full Issue Rx-to-OTC Switch/Word Count: 1165/Article #05150803005/Posted: Aug. 3, 2015 12:05 AM.)

As an example of a dangerous risk of continuing to use a statin without having real-time diagnostic test data available to the pharmacist at the point of sale, please note the following: About one in 10,000 patients can have a dangerous complication, rhabdomyolysis, that involves the breakdown of muscles. It almost always occurs in patients who were also taking drugs that interact with statins, including a class called fibrates that interfere with the body's elimination of statins. The condition can be detected with a blood test for a muscle enzyme, creative kinase, that is released by damaged muscles and when it occurs patients must stop taking statins immediately.

Further, the FDA as a general matter is also concerned about powerful Rx drugs being used for off-label indications once switched to OTC status. In the case of statins, physicians know that statins can be used to treat rheumatoid arthritis, an unapproved use. Many patients as well know this since the drugs have been prescribed for many years now and this information is broadly available on the Internet. So, another FDA concern is that unless there is a retail healthcare intermediary blocking/preventing off-label use by using real-time diagnostic data to validate a condition and control the sale of the product, patients suffering from an off-label condition, such as rheumatoid arthritis in the case of statins, might be inclined to purchase an OTC drug with no intention of using it for the labeled condition. In a purely self-service environment for a switched cholesterol medication, this will always be an issue.

Therefore, it should be clear from the above discussion on lack of patient self-compliance with a “self-service” label, the potential for dangerous side effects that can be avoided in real-time using POS diagnostic tests, and the potential for off-label use when no healthcare personnel are involved, that the invention described herein is one unique way to safely and effectively switch Rx chronic care drugs for labeled asymptomatic conditions to OTC status. There simply is no substitute (other than a physician) for the presence of a pharmacist or pharmacy personnel reviewing and using biometric data generated at POS by an approved companion diagnostic for creating true patient compliance, and for insuring that a patient receives the intended, approved benefit from the product without undue risk.

Importantly, the U.S. FDA announced a public hearing on Feb. 28, 2012 (after the priority date for the present invention) for a “new paradigm to expand Rx switches” which fundamentally suggested as feasible the type of condition-of-use label described above. Below is a quote from the document entitled: DEPARTMENT OF HEALTH AND HUMAN SERVICES, Food and Drug Administration [Docket No. FDA-2012-N-0171], Using Innovative Technologies and Other Conditions of Safe Use to Expand Which Drug Products Can Be Considered Nonprescription; Public Hearing.

“FDA is considering whether medications for certain diseases or conditions that would otherwise be available only by prescription could be made available without a prescription with certain conditions of safe use. For example, some conditions of safe use could be designed to assist patients in self-selection of an appropriate medication or provide for follow up monitoring during continued use. The conditions of use could include requiring pharmacist intervention to ensure appropriate nonprescription use. Additionally, conditions of safe use could involve the use of innovative technologies, such as diagnostics approved or cleared by FDA for use in the pharmacy or other setting.”

Accordingly, it is clear from the FDA announcement that this type of label had not been considered feasible from a regulatory standpoint by the FDA prior to the announcement. In effect, the FDA, as the industry expert, has discouraged drug manufacturers from submitting a condition-of-use label using a companion diagnostic test as a way to gain approval for an Rx-to-OTC switch of a chronic care drug for an asymptomatic condition.

This new FDA initiative is designed to resolve an unmet long-standing, public health need to improve consumer access to certain chronic care drugs, such as cholesterol reduction, diabetes, and hypertension medications, all of which could have a significant and long-lasting beneficial effect in patient populations who might otherwise not have access to these drugs either because of low awareness/ignorance of the diseases, the lack of health insurance, or access to healthcare professionals. In the case of statins, elevated cholesterol levels are one of the most significant risk factors involved with Coronary Heart Disease (“CHD”) and are associated with numerous cardiovascular events. A significant public health benefit could be derived if these medications were accessible in a more convenient, more widespread OTC purchase environment. Further, if prescription drugs for chronic conditions can be made more broadly and more inexpensively available with appropriate safeguards, then the population will have better access to these drugs and be healthier overall. Lastly, the health care system will be less burdened from an expense standpoint because of fewer patients being admitted to emergency rooms for treatment of conditions that could be controlled by more readily available OTC pharmaceuticals.

### OBJECTS AND SUMMARY OF THE INVENTION

It is therefore an object of the present invention to provide a viable regulatory manner of switching certain drugs using a combination product label from prescription-only status to behind the counter (“BTC”) or over-the-counter (“OTC”) status by designating a specific CLIA-waived diagnostic test and its real-time results to be assessed by retail healthcare personnel in the context of the drug's label as the mandatory condition-of-use before the drug can be offered for sale.

It is a further object of the present invention to provide methods to make sure initially the patient has the disease parameters for which the product is intended in order to insure the patient is purchasing the drug for the labeled indication and because of the involvement of pharmacy personnel is blocked/prevented from purchasing the drug for an off-guideline labeled indication as well as an off-label indication/use.

It is a further object to provide methods to make sure subsequent to drug use to treat an asymptomatic chronic disease state or condition in human patients that the companion diagnostic as part of the combination label is used a second time by pharmacy personnel to insure that the individual patient is actually receiving a benefit from the drug and that undue side effects or potential serious adverse events are not an issue.

It is a further object of the invention to provide a method for repurchasing an Rx-to-OTC switched drug subject to a condition-of-use drug label via a method which allows for the evaluation of the efficacy and safety of the drug regimen with the cooperation of a retail healthcare person.

It is a further object of the invention to provide a method for having only a specific CLIA-waived test designated on the condition of use label to be administered only by trained retail healthcare persons in a Food/Drug/Mass Merchandiser environment that has obtained a valid CLIA certificate of waiver to be able to administer such tests.

In accordance with the above objects and others which will be apparent to persons having ordinary skill in the art, the present invention is directed in part to a method of making available a medication over-the-counter to human patients in need thereof, comprising administering a diagnostic test to human patients by trained healthcare personnel at a retail point of sale location with a valid CLIA certificate of waiver using a point-of-sale, CLIA-waived diagnostic machine as designated on the drug label in order to screen the patients and determine whether the patients have disease conditions or biomarkers which indicate the suitability of drug therapy for a particular disease, wherein the drug has a condition-of-use drug label approved by a regulatory authority which authorizes over-the-counter availability of the drug upon a determination at the point of sale by trained healthcare personnel that the patients are in need of treatment with the drug based on the results of the diagnostic test; and offering to the patients a drug regimen comprising a plurality of unit doses of a suitable dosage of a drug for the treatment of a particular disease if the result of the diagnostic test indicates that the patients would benefit from treatment with the drug.

In certain preferred embodiments, the method further comprises utilizing a retail healthcare person to review and analyze the results of the diagnostic test only in the context of the approved drug label in order to make a sale or no sale decision. The pharmacist uses the guidelines described on the drug label along with his or her professional training with respect to the diagnostic test results generated and in no way relies on algorithms or stored or transmitted data on generic computers to make an informed decision.

In certain preferred embodiments, the method further comprises providing the patients who would benefit from treatment with the drug with a random access code, so that the patients having the random access code can enter the code into a lockable, over-the-counter dispensing cabinet which will then dispense said plurality of unit doses of said drug to the patients should the patients decide to purchase the same.

In other preferred embodiments, the method further comprises having the retail healthcare person offer the drugs to the patient(s) from behind-the-counter, such that the patient can accept the drug regimen and purchase the same, or decline to purchase the same.

In certain preferred embodiments, the results of the diagnostic tests of the human patients are entered into a national patient registry as proof of the drug's effectiveness or lack thereof so that the patients who would benefit from treatment with the drug can purchase the drug at a different retail point of sale location which has access to the national patient registry.

In certain preferred embodiments, the method further comprises advising the patient(s) that they must be administered a follow-up test prior to receiving a refill of the drug regimen.

The method preferably also includes administering to the said patients who receive the drug regimen a second diagnostic test at a retail point of sale location for drugs using the point-of-sale, CLIA-waived diagnostic machine designated on the drug's label in order to determine whether the patient's condition is being adequately treated with the dosage regimen and/or whether the patient is experiencing any problematic side effects, and dispensing a second drug regimen to the patient if the result of the diagnostic test indicates that the patient would continue to benefit from treatment with the drug. Preferably, the patient who would continue to benefit from treatment with the drug is provided with the ability to obtain long-term refills of the drug regimen at retail point-of-sale locations having access to the national patient registry.

In certain preferred embodiments, the invention is further directed in part to methods to make sure subsequent to drug use to treat an asymptomatic chronic disease state or condition in human patients that the companion diagnostic as part of the combination label is used a second time by pharmacy personnel to insure that the individual patient is actually receiving a benefit from the drug and that undue side effects or potential serious adverse events are not an issue.

In certain preferred embodiments, the invention further allows healthcare personnel to make sure initially that the patient has the disease parameters for which the product is intended in order to insure the patient is purchasing the drug for the labeled indication and because of the involvement of pharmacy personnel is blocked/prevented from purchasing the drug for an off-guideline labeled indication as well as an off-label indication/use.

In certain preferred embodiments, the invention relates in part to a method for repurchasing an Rx-to-OTC switched drug subject to a condition-of-use drug label via a method which allows for the evaluation of the efficacy and safety of the drug regimen with the cooperation of a retail healthcare person.

In certain preferred embodiments, a specific CLIA-waived test is designated on the condition of use label to be administered only by trained retail healthcare persons in a Food/Drug/Mass Merchandiser environment that has obtained a valid CLIA certificate of waiver to be able to administer such tests.

The above methods are particularly useful where the drug regimen is suitable for treatment of a chronic disease state, (e.g., an asymptomatic chronic disease state) or where serious side effects can be identified with POS fluid-based diagnostic tests and which can be adequately treated on an over-the-counter basis under a condition-of-use drug label utilizing data obtained from a specified point-of-sale, CLIA-waived diagnostic machine and evaluated by a retail healthcare person in the context of the approved drug label. Such drugs for chronic disease states and conditions include, but are not limited to, e.g., a drug for cholesterol reduction, a drug for diabetes control, a drug for medical obesity, a drug for control of hypertension, and a drug for treating benign prostatic hyperplasia. Examples of such drugs include, without limitation, lovastatin, simvastatin, pravastatin, atorvastatin, metformin, pioglitazone, liraglutide, sitagliptin, saxagliptin, canagliflozin, chloropropamide, glimepiride, glyburide, tolazamide, tolbutamide, a combination of phentermine and topiramate, lorcaserin, valsartan, metoprolol, atenolol, propranolol, carvedilol, amlodipine, olmesartan meloxomil, losartan, irbesartan, a combination of amlodipine and benazepril, aliskiren, a combination of hydrochlorothiazide and losartan, ramipril, lotensin, captopril, enalapril, fosinopril, lisinopril, moexipril, perindopril, quinapril, trandolapril, candesartan, telmisartan, diltiazem, terazosin, prazosin, alfuzosin, finasteride, dutasteride, tamsulosin, and doxazosin.

In certain preferred embodiments, the CLIA-waived diagnostic machine comprises an analyzer, further comprising inserting into the analyzer a disc containing various reagents along with a fluid sample comprising a drop of blood or saliva from the patient, the machine and the disc together being capable of analyzing the fluid sample for a particular disease or condition.

The patients who receive the drug regimen are administered a second diagnostic test by a retail healthcare person at a retail point of sale location for drugs (e.g., when seeking a refill of the drug) using the CLIA-waived diagnostic machine in order to determine whether the patient's condition is being adequately treated with the dosage regimen and/or whether the patient is experiencing any problematic side effects, wherein the diagnostic test is capable of analyzing a second fluid sample taken from the patients in order to determine the patient's response or lack thereof to the drug as well as other biomarkers to determine asymptomatic side effects. The second drug regimen (meaning the refill) is offered to the patient if the result of the diagnostic test indicates that the patient would continue to benefit from treatment with the drug. In certain preferred embodiments, the patient then accepts the drug regimen, which may be provided to the patient, e.g., via the dispensing cabinet or given to the patient by a retail healthcare person.

The invention is further directed to a method of treating human patients on an over-the-counter basis with a drug typically dispensed to a patient only with a prescription from a physician, comprising obtaining a condition-of-use OTC drug label which designates a specific point-of-sale, CLIA-waived diagnostic test for diagnosing a disease state or condition in human patients, administering the CLIA-waived diagnostic test to a human patient at a retail point of sale location with a valid CLIA certificate of waiver by trained healthcare personnel, and offering to the patient a drug regimen comprising a plurality of unit doses of a suitable dosage of a drug for the treatment of a particular disease if the result of the diagnostic test indicates that the patient would benefit from treatment with the drug per the approved indication on the drug's label. In this method, the CLIA-waived diagnostic machine must be used at the point of sale as directed by the drug label in order to screen the patients and determine whether the patient has disease conditions or biomarkers which indicate the suitability of drug therapy for a particular disease and to prevent/block off-guideline labeled use and off-label use. A retail healthcare person must analyze the results of the diagnostic test consistent with the requirements of the retail location's CLIA certificate of waiver. In certain preferred embodiments, the patients who would benefit from treatment with the drug are provided with a random access code, so that the patient having the random access code can enter the code into a lockable, over-the-counter dispensing cabinet which will then dispense said plurality of unit doses of said drug to the patient. Alternatively, the retail healthcare person may offer the drug from behind the counter.

In certain preferred embodiments, the results of the diagnostic tests of the human patient is entered into a national patient registry so that the patient who would benefit from treatment with the drug can purchase the drug at a different retail point of sale location which has access to the national patient registry.

In certain preferred embodiments, the method further comprises administering to said patient who receives the drug regimen a second diagnostic test at a retail point-of-sale location for drugs using a point-of-sale, CLIA-waived diagnostic machine specified on the drug label in order to determine whether the patient's condition is being adequately treated with the dosage regimen and/or whether the patient is experiencing any problematic side effects, and dispensing a second drug regimen to the patient if the result of the diagnostic test indicates that the patient would continue to benefit from treatment with the drug. In certain preferred embodiments, the patient who would continue to benefit from treatment with the drug is provided with the ability to obtain long-term refills of the drug regimen at retail point-of-sale locations having access to the national patient registry.

The invention is further directed to a method of treating human patients on an over-the-counter basis with a drug typically dispensed to a patient only with a prescription from a healthcare person, comprising obtaining a condition-of-use OTC drug label which designates a specific point-of-sale, CLIA-waived diagnostic test for determining whether a human patient's condition is being adequately treated with the drug and/or whether the patient is experiencing any problematic side effects with said drug, wherein the condition-of-use drug label requires the administration of a diagnostic test by trained healthcare personnel at a retail point-of-sale location with a valid CLIA certificate of waiver using a specific designated point-of-sale, CLIA-waived diagnostic machine in order to determine whether the patient would continue to benefit from treatment with the drug, which would allow a patient who would continue to benefit from treatment with the drug the ability to obtain long-term refills of the drug on an OTC basis.

Preferably, the method further comprises administering to said patients who have received the drug a diagnostic test at a retail point-of-sale location with a valid CLIA certificate of waiver using a point-of-sale, CLIA-waived diagnostic machine as designated on the drug's label in order to determine whether the patient would continue to benefit from treatment with the drug. Preferably, one or more refills of the drug may be provided to the patient if the result of the diagnostic test indicates that the patient would continue to benefit from treatment with the drug.

The method may further comprise initially prescribing a drug regimen comprising a dosage of a drug administered at suitable intervals for a human patient through a physician visit in which the physician examines the patient and makes a diagnosis for a disease state or condition in the patient which is treatable with that drug. The results of the diagnostic test of the human patient may be entered into a national patient registry so that the patient can purchase the drug at a different retail point of sale location which has access to the national patient registry, if the diagnostic test determines that the patient would benefit from continued treatment with the drug.

The method may further comprise entering the results of the diagnostic test of the human patient into a national patient registry so that the patient can purchase the drug at a different retail point-of-sale location which has access to the national patient registry, if the diagnostic test determines that the patient would benefit from continued treatment with the drug.

The method may further comprise providing the patients who would continue to benefit from treatment with the drug with the ability to obtain long-term refills of the drug regimen at retail point-of-sale locations having access to the national patient registry.

In certain preferred embodiments, the CLIA-waived diagnostic machine comprises an analyzer, further comprising inserting into the analyzer a single-use test panel on a disc along with a fluid sample comprising a drop of blood or saliva from the patient, the test panel and diagnostic machine being capable of analyzing the fluid sample for a particular disease or condition.

In certain preferred embodiments of any and all of the above methods, the drug is in the form of an oral solid dosage form.

In certain preferred embodiments of any and all of the above methods, a retail healthcare person analyzes the results of the diagnostic test and makes the decision (based on those results in the context of the approved drug label) as to whether the patient should be administered (or re-administered) the drug regimen. In other words, the approved drug label will contain information concerning the tests results that would be obtained from the diagnostic test, and the healthcare person will compare the test results obtained from the diagnostic test to the guidelines provided in the approved drug label to make the determination as to whether the patient is a candidate for treatment with the drug (e.g., whether the test results fall within the treatment guidelines in the approved drug label that indicate that the patient is suffering from the condition in question). If the test results fall within the guidelines set forth in the approved drug label, then a healthcare person can offer the drug for sale. If the test results fall outside of the treatment guidelines, then the healthcare person can determine that the drug is not appropriate to be sold to the patient and therefore would not offer the drug for sale to the patient.

The invention is further directed to a method of administering an over the counter drug for treatment of a (e.g., asymptomatic) chronic medical condition to a patient comprising providing a drug label for over-the-counter treatment of a chronic medical condition, said drug label requiring a new patient to take an initial point-of-service diagnostic test to determine whether the patient suffers from the condition treated by the drug and requiring patients already receiving the drug on an over-the-counter basis to take one or more follow up point-of-service diagnostic tests to determine whether the patient is benefiting from the drug; providing a patient who is determined eligible to take the drug with a random access code for use in association with a lockable, electromechanical cabinet to receive an appropriate dose of the drug to treat the patient's chronic medical condition. Preferably, the method further comprises providing a follow up point-of-service diagnostic test which determines whether the patient is experiencing any undue side effects from the drug. The method further preferably comprises creating and maintaining a national patient registry for containing the point of service diagnostic test results, which can be accessed by appropriate health care persons as proof of patient eligibility.

As used herein, the terms “over-the-counter” or “OTC” or “non-prescription medicine” refer to drugs that may be purchased (e.g., by a patient) without a prescription from a retail outlet such as a Food, Drug, or Mass Merchandiser retail store. All these terms are used synonymously herein.

As used herein, the term “behind-the-counter” or BTC medicine refers to drugs which may be purchased at the pharmacy without seeing a doctor. Unlike OTC drugs, however, access would not be allowed without the intervention of a learned intermediary. But, unlike a prescription medication, BTC status would allow a patient to access drugs after an assessment and decision by a pharmacist. Similar to over-the-counter (OTC) status, BTC would allow a patient to access medications without seeing a doctor. Therefore, the terms “over-the-counter”, or “OTC” or “non-prescription medicine” are, for the purposes of the present invention, deemed to include BTC medicine.

As used herein, the terms “biometric data” and “biometric characteristics” are used interchangeably and shall collectively mean measured data representative of one or more physiological conditions of the purchaser. Such “biometric data” and “biometric characteristics” can be measured in real time via the use of a CLIA-waived, diagnostic apparatus such as that described herein (e.g., such as that commercially available from Abaxis via its Piccolo Express machine), and may include the testing of a fluid sample from the human patient, e.g., a saliva or blood sample (e.g., whole blood, serum). In addition or alternatively, the biometric data can be further obtained from the human patient via for example, a blood pressure cuff, a thermometer, an infrared blood gas analyzer, and the like. The biometric data can be limited to one or more of such samples taken from the patient, or may include all of such samples. The biometric data may be further supplemented by information obtained from the human patient by the retail healthcare person, such as height, weight, previous medical history, and any combinations thereof.

As used herein, the term “retail healthcare person” may encompass a trained (human) technician, a pharmacist, a nurse, or other person trained in healthcare and having specific knowledge and understanding of the present methods described herein, the ability to understand the diagnostic test results in the context of the approved drug label, and to make a determination as to whether or not to offer the patient a drug regimen for the condition or disease state that the patient has been tested for, as well as (in certain embodiments) providing the drug regimen to the patient if the situation calls for the same.

### DETAILED DESCRIPTION OF THE INVENTION

The entire OTC pharmaceutical industry has been attempting to make prescription (“Rx”) chronic care drugs (e.g., for asymptomatic conditions) available OTC. This has led to an over-reliance on devising drug labels that could be shown to help consumers include or exclude themselves as appropriate for therapy and then further exclude themselves based on creating an awareness of the signs and symptoms associated with potentially severe side effects. This approach has resulted in the FDA disapproving all applications to switch prescription drugs for treatment of (e.g., asymptomatic) chronic conditions to an OTC status. In effect, the FDA, as the decisive expert in the industry, has encouraged these labeling-only solutions and has taught away from allowing drug sponsors to devise condition-of-use labeling solutions that rely on POS diagnostic data to require or allow pharmacy personnel to help patients select or de-select themselves.

The present invention provides sufficient controls and safeguards to enable retail healthcare professionals and technicians to functionally determine if 1) patients are suitable candidates for initially taking a drug (i.e. the patient suffers from the condition for which the drug is approved and not some off-guideline labeled condition or pure off-label condition), and 2) whether the continued use of the drug by a patient over the long term is appropriate based on the results of follow-up POS diagnostic tests which can demonstrate whether the benefits of the drug outweigh its risks. Specifically, the invention combines the following: 1) a condition of use OTC drug label which designates a specific companion point-of-sale (“POS”), CLIA-waived diagnostic test (CLIA stands for Central Laboratory Improvement Amendments; these amendments permit simple diagnostic tests to be performed outside of a central lab if results can be easily understood by a lay person and the site obtains a CLIA certificate of waiver) for diagnosing the disease state before drug use and the effectiveness and safety biomarkers after drug use, with 2) a lockable, over-the-counter (“OTC”) dispensing cabinet with random access code capabilities for controlled retail distribution to eligible patients only and for the inputting of individual patient's test results into a patient registry, and with 3) a national patient registry where the pharmacists input the patients' results so that patients can purchase a product at any retail store that has access to the patient registry. This combination of elements enables previously prescription-only chronic care drugs for asymptomatic conditions, e.g., statins for cholesterol reduction, to safely be switched to OTC status and be broadly available at all times to any eligible patients.

The novel methods and machines of the present invention are used in combination to enable certain chronic care Rx medications to be safely switched from Rx to OTC status by limiting their exposure only to patients who have been shown to suffer from the disease, are responders to the drug, do not have undue side effects which could offset the drug's benefits, and by preventing/blocking the use of the drug for off-guideline labeled use and off-label use through the involvement of a healthcare professional. Further, the patient must agree to allow their test data to be included in a national patient registry so that any retail store personnel at any location can access that data to determine if the patient is eligible to purchase the drug at retail for subsequent use after the initial purchase regardless of whether or not that retail location has a CLIA certificate of waiver to conduct the specific CLIA-waived diagnostic test specified on the drug label.

One example of how the method works is as follows:
1. The patient sees advertising or some other promotional effort making them aware that there is now an OTC drug for a chronic condition available at food, drug, and mass merchandiser stores without a prescription. But they must take a quick diagnostic test administered by local pharmacy personnel to determine if the drug is right for them.
2. The patient goes to a retail setting and asks the pharmacy to conduct the required diagnostic test. Any trained health care personnel would be allowed to conduct the test because of the CLIA-waived status of the diagnostic test equipment.
3. Within 15 minutes or less the patient receives their test results and is informed whether they have met the conditions set by the FDA on the drug's label to be eligible to receive the drug.
4. If the patient is eligible they are given a randomized code and told to use this code in the OTC dispensing cabinet right next to the pharmacy. If the patient is not eligible, they are informed that they do not currently suffer from the condition and do not need the drug.
5. The eligible patient takes the randomized code to the machine, enters the code, and receives the medicine. The health care person may enter the patient's information into the system which transmits this information on an encrypted basis, thereby providing the patient with the ability to use the randomized code in the machine either for the initial or subsequent purchases. Alternatively, the pharmacist offers the product for sale and also enters the patient's test information directly into the patient registry. The patient then pays for the medicine at checkout along with all their other store purchases, or alternatively makes a credit or debit card payment directly at the machine.
6. The drug's label informs the patient that they must return to the store for a follow-up test to make sure the drug is actually working and that there are no asymptomatic side effects. If they then pass this second test again administered by pharmacy personnel, they will receive a code that is good for a set number of refills per the drug's label.

This paradigm has never before been used for an Rx-to-OTC switch of any Rx drug. This is because the FDA has historically worked under the principle that OTC drugs should be used for conditions that are patient-diagnosable, drugs that are patient-selectable in terms of the proper medication and dosage for purchase at retail without the advice of store personnel, and patient-treatable at home. Nevertheless, there are exceptions to this principle but none of the exceptions utilize the aforementioned methods and elements to permit OTC availability. (For example, one such labeling exception is that the label requires that the patient receive an initial diagnosis for the condition, a yeast infection, from a physician before purchasing an antimicrobial for that condition. However, there are no retail or labeled controls to prevent the consumer from purchasing the product in the absence of such diagnosis.)

A partial, non-limiting list of Oral, Chronic Care Drugs Potentially Suitable for Switching Under the Proposed Methods includes the following drugs in the following categories that would benefit from this switch paradigm:
Cholesterol Reduction: lovastatin, simvastatin, prevastatin, fluvastatin, rosuvastatin, and atorvastatin
Diabetes Control: metformin, pioglitazone, liraglutide, sitagliptin, saxagliptin, and canagliflozin
Medical Obesity: phenterminettopiramate, and lorcaserin
Hypertension: valsartan, metoprolol, amlodipine, olmesartan, losartan, irbesartan, amlodipine, aliskiren, telmisartan, and diltiazem
Benign Prostatic Hyperplasia: terazosin, dutasteride, tamsulosin, and doxazosin

The specific elements that are used in combination are as follows:

Condition of Use Label: For this element, the drug manufacturer must show through controlled clinical trials that their drug can be effectively and safely used based on an initial and follow up diagnostic report by a POS, CLIA-waived diagnostic machine designated on the drug's label (an example of which is specifically identified as the Abaxis Piccolo Xpress). For approval by the FDA this would involve the submission of clinical trial data that prove statistically that patients with and without the disease can be identified by particular CLIA-waived equipment to be used at retail (evidence of diagnostic specificity and sensitivity), that patients with the disease who are treated with the drug can be subsequently screened by the same diagnostic equipment to show that the patient's disease is being treated by the drug or not (evidence of effectiveness), and that the patient is not expressing any biomarkers that would indicate problematic side effects (evidence of safety). In such a label, both the drug and the specific diagnostic test equipment would appear on the drug's label requiring that they both be used in conjunction with each other. Those patients that pass the two phases of diagnostic screening administered by pharmacy personnel would be eligible for long-term drug refills.

Diagnostic Test Equipment: Recently, new table-top diagnostic test equipment (the Piccolo Xpress) has been developed by Abaxis, Inc. of Union City, Calif., for use at the Point of Sale or Service (POS) with physicians. The results from this machine can be delivered in under 15 minutes making its use practical for the first time in determining if a patient meets the requirements for receiving a drug under the proposed, Rx-to-OTC switch treatment method patent. Because of these rapid assay tests and their relatively low cost, there is no longer the customary delay in getting diagnostic test results as there has been with historical tests, which required that biological samples be sent to a central lab for analysis and reporting. These results could take days to be reported. Importantly, it should be noted that these tests have always been used for the diagnosis of Rx conditions leading to the prescribing of Rx drugs and that they have never been used as part of either an Rx or OTC drug combination product label where a drug is linked to a device or diagnostic and vice versa. As a result these types of diagnostic tests have never been relevant to the OTC pharmaceutical industry, which is the relevant industry for the invention described herein.

The diagnostic test equipment, as an example, may comprise an analyzer, which is a table top piece of equipment, and various Abaxis metabolic test “panels” or discs which are inserted into the analyzer along with a drop of blood or saliva and are good for a single use. These test panels are designed to read in under 15 minutes a fluid sample for particular disease conditions and other biomarkers indicative of the patient's response or lack thereof to a drug as well as other biomarkers to determine asymptomatic side effects. Therefore, in certain embodiments, the patient's biomarkers will determine whether the patient suffers from the condition treated by the drug. The label, as noted above, would also require patient follow-up with the same POS diagnostic test equipment but with the same or perhaps different test panel conducted to insure that the patient is benefiting from the drug by a reduction or increase, as the case may be, in the same biomarkers. In addition, in certain embodiments, the label would additionally require patient follow-up with this POS diagnostic test to determine whether there are any undue side effects, particularly those that cannot be detected by the patient.

Roughly the size of a shoebox, Rx health care providers use the Piccolo Xpress' easy-to-follow color touch screen commands to perform routine multi-chemistry panels (A test panel is a predetermined group of medical tests as an aid in the diagnosis and treatment of disease). 0.1 cc of whole blood, serum or plasma is added directly to the patented 8-cm diameter single use plastic disc containing the liquid diluent and dry reagents. The disc is placed in the analyzer drawer where centrifugal and capillary forces are used to mix the reagents and sample in the disc. The Piccolo Xpress monitors the reagent reactions simultaneously using nine wavelengths, calculates the results from the absorbance data and reports the results. The Piccolo Xpress self-calibrates with each run and the on-board continuous intelligent Quality Control (iQCT) monitors the analyzer, reagent reactions and sample to ensure chemistry and instrument integrity. This assures the user the highest quality and accuracy in results. Results are ready in approximately 12 minutes with a “hard copy” report suitable for the patient file or transferred to a computer, printer or an LIS/EMR. This enables the user to rapidly obtain critical diagnostic information at point of care. The easy-to-use, low maintenance system requires no special training to operate.

The present invention is not limited by any particular diagnostic test equipment, and one skilled in the art will appreciate that currently available diagnostic equipment other than that which is specifically described herein may be used or modified to be used in conjunction with the present invention, and such modifications are deemed to be within the scope of the appended claims.

The diagnostic test equipment is preferably arranged to analyze a sample from the human patient, e.g., a saliva or blood sample (e.g., whole blood, serum), and test that sample for known parameters indicative of a particular chronic condition or disease state which the patient suspects that they may be suffering from. In addition or alternatively, biometric data can be obtained from the human patient via for example, a blood pressure cuff, a thermometer, an infrared blood gas analyzer, and the like. Additional information questions regarding the identity of the customer may also be asked, e.g., by the retail healthcare person, such as billing information, address, age, gender, previous relevant medical history, symptoms, etc., or for previous users of the system this information may be loaded from an associated database. The information may be obtained via a computerized medical questionnaire, which may be interactive, with certain answers provided by the patient determining whether and which additional questions will be asked. The questions may also be formulated by the retail healthcare person/professional.

Retail Dispensing Machine: A patient eligible to take the medication based on the results of their initial and follow-up POS tests may be given a random access code for a lockable, electromechanical OTC cabinet in order to receive the appropriate pharmaceutical product and dose to treat the condition from which they suffer. This OTC dispensing cabinet may be placed in a normal retail location but sufficiently near an electrical outlet for automation purposes. It would preferably have a feature for entering a random code such as a keypad or touch pad and it would be of adequate size to be able to hold enough inventory to meet daily demand. It may preferably also be designed with the capability to accept individual patient test data and transmit it wirelessly on an encrypted basis to the national patient registry database. It may also be designed with the ability to take a credit card to enable the patient to be able to pay for the medicine at the time it is dispensed from the machine.

A wide variety of apparatus are used in healthcare facilities for the dispensing and inventory of medications and medical supplies are known, including, for example, those described in U.S. Pat. No. 5,520,450 (a supply station with an internal computer; the supply station is comprised of a cabinet having a plurality of lockable doors); other examples of computer controlled dispensing apparatus are found in U.S. Pat. Nos. 5,346,297, 5,905,653 and 5,745,366, all of which are hereby incorporated by reference. Also, Japan Patent 10334326 describes an automatic vending machine having a computer with means for storing and processing information on “general medicines or the like”. The AcuDose-Rx dispensing cabinet available from McKesson Automation Inc. of Pittsburgh, Pa. is an example of a computer-controlled cabinet for dispensing medications. A person having ordinary skill in the art can adapt any of these devices and/or other known devices for use in conjunction with the herein-disclosed methods.

Communication between the (e.g., retail) healthcare person's computer and/or the diagnostic test apparatus and the lockable cabinet may be over a communication network that may be, for example, a wired network or a wireless communication network, or a computer network such as the Internet, an Intranet or a local access network (LAN). The connected devices may further be in communication with a central computer system that, in turn, communicates with prescription providers, insurance companies, the national registry, and/or the like.

National Patient Registry/Database: In certain embodiments of the present invention, a national patient registry is created where the POS diagnostic test results are received from the retail dispensing machine and then contained for access by any other retail location. In a further embodiment, the test results can be transmitted by the diagnostic machine and contained on a suitable secure electronic database or electronic computer disks. In certain embodiments, the test results can be accessed by, e.g., pharmacists, pharmacy technicians and/or other retail health care professionals through the national pharmaceutical claims adjudication systems that all pharmacies subscribe to. In other embodiments, other suitable means for access by appropriate health care professionals are made available, such as access by a secure Internet site. This registry permits universal access to the medicines by patients who are shopping locally or are travelling.

### Theoretical Example of a Chronic Care Drug for Switching Under the Proposed Methods

As described above, Mevacor® has been proposed for switching to OTC status by Merck on three separate occasions. In each instance Merck has proposed detailed labeling changes which were to be considered by the patient for self-selection and de-selection prior to purchasing Mevacor OTC. These attempts were assessed by the FDA and were considered to be inadequate.

There were various issues that were considered by the FDA including whether or not consumers would understand based on the label whether they would benefit from the drug and would they understand based on the label if they were experiencing undue side effects such as elevated liver enzymes. Since both the benefit and some critical risks were asymptomatic, it was not clear to the FDA how a label alone with no controls at retail could possibly have allowed sufferers to make an intelligent initial purchase decision or the subsequent decision to repurchase the drug or, alternatively, to restrict them from making the purchase decision.

Below is part of the proposed Mevacor label from Merck's New Drug Application of 2007—

Before buying:
You must have tried a healthy diet and exercise to reduce your cholesterol.
You must have had a fasting cholesterol test and know your cholesterol numbers.
Your Total cholesterol must be 200 to 240.
Women must also have HDL “good” cholesterol 1 to 59.

Using the present invention, if a Mevacor label were created which outlined all conditions of safe use and provided the necessary means to meet those conditions including a POS, real-time diagnostic test, then it should be quite clear who would be eligible for Mevacor on both an initial and repeat basis. There simply would be no reason to not know one's numbers or to misremember the numbers. Then, there would have to be some controlled way, by having the pharmacy personnel administer the CLIA-waived diagnostic test, to make sure the consumer could not purchase the drug if the benefit was not present and/or the risks were too great and to make sure the patient was not purchasing the product for some unapproved use. The present invention is a unique way to provide both the necessary and sufficient safeguards to permit OTC usage of such a drug as Mevacor by allowing consumers and retail personnel to know in real time all the information required on the drug's label before a valid purchase can be made.

With this invention the Mevacor label could read in part and would also be displayed on the dispensing cabinet.

Before You can Purchase This Drug:
You must have tried a healthy diet and exercise to reduce your cholesterol.
You must take a rapid assay diagnostic test administered by pharmacy personnel in order to determine your cholesterol levels; this test takes only 12 minutes and is free to the patient.
The test must indicate that your total cholesterol is within the required 200 to 240 range.
The test must also indicate that women must have HDL “good” cholesterol from 1 to 59.
You must return for a follow up diagnostic test also administered by pharmacy personnel to insure that you are receiving a benefit from the drug and that there are no asymptomatic side effects.

### CONCLUSION

While the present invention has been described in conjunction with preferred embodiments thereof, those of ordinary skill in the art will recognize that many modifications and variations may be implemented while still falling within the scope of the present invention. For example, processing techniques using methods and an apparatus other than the CLIA-waived Abaxis diagnostic test equipment specifically described herein may be utilized, and are contemplated to be within the scope of the appended claims. Other process steps may be substituted for those specifically described herein while remaining within the scope of the present invention. Those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the appended claims.

What is claimed is:

1. A method of self-treating a human patient for a chronic disease comprising:
self-administering to the human patient in need a unit dose of a pharmaceutically acceptable drug in a sufficient amount to treat a chronic disease of the patient;
wherein prior to self-administration of the drug, a CLIA-waived diagnostic test was administered to the patient by having the patient provide a fluid sample to a third party to obtain approval to obtain the drug for self-administration of the chronic disease on an OTC basis via a condition-of-use instructions concerning analysis of the CLIA-waived diagnostic test results;
wherein the fluid sample was tested in the CLIA-waived diagnostic to determine whether the patient qualifies for OTC treatment with the drug;
wherein when the patient qualifies for OTC treatment of the drug by virtue of a positive result of the CLIA-waived diagnostic test indicating that the patient would benefit from treatment with the drug, prior to self-administration of the drug, the patient receives a plurality of unit doses of the drug in sufficient amount to treat a chronic disease of the patient such that the patient can self-administer a unit dose and the chronic disease state is treated.

2. The method of claim 1, wherein after all of the received unit doses have been self-administered, the patient undergoes a second CLIA-waived diagnostic test OTC to determine whether the patient is either: (i) expressing a biomarker that indicates that the patient's condition is being treated by the drug or (ii) expressing a biomarker that indicates that the patient is experiencing an asymptomatic and problematic side effect of the drug;
wherein the patient that expresses biomarkers that confirm that the patient is being treated by the drug and is not expressing the biomarker that indicates the problematic side effect, receives a second plurality of unit doses of the drug in sufficient amount to treat the chronic disease of the patient.

3. The method of claim 2, wherein the patient self-administers a unit dose of the drug from the second plurality of unit doses.

4. The method of claim 3, wherein the patient continues to undergo CLIA-waived diagnostics to determine that the patient is being treated by the drug and is not expressing the biomarker that indicates the problematic side effect and the patient continues to self-administer the drug for treatment of the chronic disease only after CLIA-waived diagnostic confirms that the patient is being treated by the drug and is not expressing a biomarker that indicates the problematic side effect.

5. The method of claim 1, wherein the chronic disease is selected from the group consisting of high cholesterol, diabetes, obesity, hypertension, and benign prostatic hyperplasia.

6. The method of claim 1, wherein the chronic disease is an asymptomatic chronic disease.
